(19) United States
(12) Patent Application Publication
(10) Pub. No.: US 2020/0246432 A1
(43) Pub. Date: Aug. 6, 2020

(54) NITRIC OXIDE- AND FAS LIGAND- ELUTING COMPOSITIONS AND DEVICES AND METHODS OF TREATMENT USING SAME

(71) Applicant: YALE UNIVERSITY, New Haven, CT (US)

(72) Inventors: Mehmet Hamdi KURAL, Hamden, CT (US); Liqiong GUI, Irvine, CA (US); Laura Elizabeth NIKLASON, Greenwich, CT (US); William Mark SALTZMAN, New Haven, CT (US)

(21) Appl. No.: 16/639,801

(22) PCT Filed: Aug. 20, 2018
(86) PCT No.: PCT/US18/47097

§ 371 Date: Feb. 18, 2020

Related U.S. Application Data
(60) Provisional application No. 62/548,165, filed on Aug. 21, 2017.
(60) Provisional application No. 62/587,821, filed on Nov. 17, 2017.

(51) Int. Cl. A61K 38/19; A61K 9/00; A61K 47/42; A61K 47/34; A61K 33/00; A61F 2/90; A61L 31/16; A61L 31/10; A61L 29/16; A61L 29/08

(52) U.S. Cl. CPC A61K 38/191; A61K 9/0024; A61K 47/42; A61K 47/34; A61K 33/00; A61F 2/90; A61M 25/104; A61L 31/10; A61L 29/16; A61L 29/085; A61M 2202/0275; A61F 2250/0067; A61L 31/16

(57) ABSTRACT

The present invention provides compositions and devices that release (a) a nitric oxide (NO) donor and/or NO, and (b) an agent that aggregates and/or trimerizes the Fas receptor, such as but not limited to a Fas-ligand (FasL). In certain embodiments, the invention includes a stent that elutes (a) a NO donor and/or NO, and (b) FasL. In other embodiments, the invention includes methods of treating a condition, such as intimal hyperplasia, in a subject by administering a stent that releases (a) a NO donor and/or NO, and (b) FasL.

### CROSS-REFERENCE TO RELATED APPLICATIONS

This application claims the benefit of priority under 35 U.S.C. § 119(e) to U.S. Provisional Patent Application No. 62/548,165, filed Aug. 21, 2017, and U.S. Provisional Patent Application No. 62/587,821, filed Nov. 17, 2017, the contents of which are incorporated by reference herein in their entirety.

### STATEMENT REGARDING FEDERALLY SPONSORED RESEARCH OR DEVELOPMENT

This invention was made with government support under HL118245 and HL127386 awarded by the National Institutes of Health. The government has certain rights in the invention.

### BACKGROUND OF THE INVENTION

Coronary artery disease (CAD) is the major cause of morbidity and mortality in western countries. CAD is caused mainly by atherosclerosis, which corresponds to narrowing and hardening of arteries due to excessive buildup of plaque on vessel walls. Invasive percutaneous coronary intervention (PCI) procedures (such as atheroctomy, balloon angioplasty, and stent deployment) restore the blood flow in diseased coronary arteries. However, one of the major drawbacks of this procedure is in-stent restenosis, where the vessel undergoes re-narrowing as a response to wall injury and endothelial denudation. Following stent deployment, endothelial denudation and exposure of the vessel wall to blood flow results in immediate platelet and fibrinogen adherence to the vessel wall, followed by adhesion of leukocytes. In addition to circulating cells adhering to the lumen, vessel wall injury due to the distension causes proliferation of medial smooth muscle cells (SMCs) and adventitial cells, with subsequent migration to the lumen surface.

Currently marketed drug-eluting stents (DES) deliver anti-proliferative drugs, mammalian target for rapamycin (mTOR) inhibitors (sirolimus, everolimus, biolimus A9, or zotarolimus), microtubule inhibitors (paclitaxel), or calcineurin blockers (tacrolimus or pimecrolimus) to the vessel injury site. This reduces in-stent restenosis, while avoiding systemic toxicity. However, the non-specific anti-proliferative effect of eluted drugs affects not only vascular smooth muscle cells (SMCs) but also vascular endothelial cells (ECs), which results in the need for prolonged anti-platelet therapy following stent deployment. The need for prolonged anti-platelet therapy arises from the growth suppression of ECs, which in turn leads to prolonged exposure of thrombogenic stent struts to the patient's bloodstream. Because DES inhibit EC growth that would otherwise cover the stent struts, the prolonged risk of clotting remains after DES deployment within a patient. This mandates long-term (often life-long) treatment with potent anticoagulants, which can create patient morbidity and mortality due to complications of the anti-coagulant therapy.

Therefore, there is an urgent need for novel stents that are chemically treated so that they release one or more chemical compounds in the area surrounding the stent, but which has a superior functional profile to current DES that are clinically available. Such chemical compounds should have differential effects on SMCs and ECs. In certain embodiments, the released one or more chemical compounds would decrease SMC viability and proliferation, without affecting EC restoration and proliferation. The present invention satisfies this need.

### SUMMARY OF THE INVENTION

In certain aspects, the present invention provides a stent capable of releasing (a) a nitric oxide (NO) donor and/or NO, and (b) an agent that aggregates and/or trimerizes a Fas receptor. In some embodiments, the agent is at least one of a Fas ligand (FasL), anti-FasR antibody, anti-FasR siRNA, camptothecin, cisplatin, curcumin, ET-18-OCH3, resveratrol, TGF-β1, etoposide, vanadate, and vinblastine. In some embodiments, the agent comprises a FasL. In some embodiments, the FasL comprises a soluble, human form of FasL. In some embodiments, the NO donor includes DetaNONOate. In some embodiments, once the stent is administered to a subject, release of (a) the NO donor and/or NO, and (b) the agent kills smooth muscle cells (SMCs) and/or macrophages, but not endothelial cells (ECs) that are proximal to the administered stent. In some embodiments, the NO donor and/or NO, and the agent are released over time. In some embodiments, the NO donor and/or NO, and the agent are released immediately.

In some embodiments, the stent comprises a coronary stent. In some embodiments, the stent is coated with a substance that releases the NO donor and/or NO, and the agent, as described herein, wherein the substance may be an ethylene-vinyl acetate copolymer (EVAc), a Poly(lactic-co-glycolic acid) (PLGA) nanoparticle, a Pluronic gel, and a protein. In some embodiments, the protein includes collagen and fibrin. In some embodiments, the substance is covalently tethered to the stent surface.

In certain aspects, the present invention provides a medical balloon capable of releasing a nitric oxide (NO) donor and/or NO, and an agent that aggregates and/or trimerizes a Fas receptor. In some embodiments, the agent includes at least one of a Fas ligand (FasL), anti-FasR antibody, anti-FasR siRNA, camptothecin, cisplatin, curcumin, ET-18-OCH3, resveratrol, TGF-β1, etoposide, vanadate, and vinblastine. In some embodiments, the agent comprises a FasL. In some embodiments, the FasL comprises a soluble, human form of FasL. In some embodiments, the NO donor comprises DetaNONOate.

In some embodiments, once the medical balloon is administered to a subject, release of the NO donor and/or NO, and the agent kills smooth muscle cells (SMCs) and/or macrophages, but not endothelial cells (ECs) that are proximal to the administered stent. In some embodiments, the NO donor and/or NO, and the agent are released over time. In some embodiments, the NO donor and/or NO, and the agent are released immediately.

In some embodiments, the medical balloon is used for balloon angioplasty.

In some embodiments, the medical balloon is coated with a substance that releases (a) the NO donor and/or NO, and (b) the agent, wherein the substance includes an ethylene-vinyl acetate copolymer (EVAc), a Poly(lactic-co-glycolic acid) (PLGA) nanoparticle, a Pluronic gel, or a protein. In some embodiments, the protein is selected from the group consisting of collagen and fibrin. In some embodiments, the substance is covalently tethered to the medical balloon surface.

In certain aspects, the present invention provides a method of treating a condition in a subject, including the step of administering to the subject a composition capable of releasing within the subject a NO donor and/or NO, and an agent that aggregates and/or trimerizes a Fas receptor, wherein the composition includes a stent, a medical balloon, a hydrophilic spacer, a polymer, or a gel. In some embodiments, the NO comprises DetaNONOate. In some embodiments, the agent comprises a FasL. In some embodiments, the FasL comprises a soluble, human form of FasL.

In some embodiments, release of the NO donor and/or NO, and the agent kills smooth muscle cells (SMCs) and/or macrophages, but not endothelial cells (ECs) that are proximal to the administered composition. In some embodiments, the composition is administered to the subject's heart, artery, vein, ureter, urethra, trachea, mainstem bronchus, bronchial airway, pyloris, duodenum, esophagus, or gastro-esophageal junction. In some embodiments, the composition is coated with a substance that releases the NO donor and/or NO, and the agent, wherein the substance includes a hydrophilic spacer, a Pluronic gel, Poly(lactic-co-glycolic acid) (PLGA), or a protein. In some embodiments, the polymer or gel is applied inside a blood vessel. In some embodiments, the protein may include collagen and/or fibrin. In some embodiments, the substance is covalently tethered to the surface of the composition.

In some embodiments, the NO donor and/or NO, and the agent are released over time. In some embodiments, the NO donor and/or NO, and the agent are released immediately.

In some embodiments, the method of the present invention treats a condition in a subject including intimal hyperplasia, restenosis, anastomosis, gastric outlet syndrome, coronary artery disease, atherosclerosis, neointimal hyperplasia, pseudointimal hyperplasia, inflammation, transplantation-induced immunity, diabetes, hypertension, and/or conditions wherein the SMCs are hypertrophic and/or hyperproliferative. In some embodiments, the subject is human. In some embodiments, the subject does not require co-administration of anti-coagulation treatment.

In certain aspects, the present invention provides a drug delivery composition that includes a NO donor and/or NO, and an agent that aggregates and/or trimerizes the Fas receptor. In some embodiments, the NO donor comprises DetaNONOate. In some embodiments, the agent comprises a FasL. In some embodiments, the FasL comprises a soluble, human form of FasL. In some embodiments, the composition is one which kills smooth muscle cells (SMCs) and/or macrophages, but not endothelial cells (ECs). In some embodiments, the composition acts locally. In some embodiments, the composition includes a polymer or a gel.

In certain aspects, the present invention provides a kit that includes the composition of as described herein, and instructional material for use thereof.

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIG. 1 is a set of images illustrating an embodiment of the stent of the present invention. Nitric oxide (NO) released/produced by the stent interacts with smooth muscle cells (SMCs) in the proximity of the stent, and thereby increases Fas receptors on the surface of these cells. Fas ligand (FasL) released by the stent binds to the receptors that are expressed on the SMC, and forms a death-inducing signaling complex in SMCs that are close to the stent and to the vessel lumen.

FIG. 2 is a set of images illustrating the finding that 24 hours of treatment with 0.1 mM DetaNONOate (NO donor) increased Fas receptors on the surface of human aortic SMCs dramatically. Blue=DAPI nuclei, red=Fas receptor. Scale bar: 50 μm.

FIG. 3 is a set of graphs illustrating the finding that 48 hours of treatment with varying concentrations of human FasL and DetaNONOate (NO donor) increased apoptosis in human (left panel) and pig (right panel) aortic SMCs in a synergistic way.

FIG. 4 is a series of graphs and images illustrating the finding that 96 hours of treatment with varying concentrations of human FasL and DetaNONOate (NO donor) did not cause apoptosis in human umbilical vein endothelial cells. NS: Not significant.

FIGS. 5A-5C are a series of images illustrating pig coronary arteries and cultured in a tissue culture dish in culture medium inside of an incubator. FIG. 5A depicts freshly isolated artery. FIG. 5B depicts artery cultured for nine days. FIG. 5C depicts artery cultured and treated with 400 ng/mL FasL and 0.1 mM DetaNONOate (NO donor) for nine days.

FIGS. 6A-6D are a series of images illustrating pig coronary arteries cultured in culture dishes in culture medium for nine days. Addition of FasL and DetaNONOate to culture medium decreased SMC intima layer thickness, and prevented vessel wall thickening. Endothelial cell viability and presence on the lumen of the arteries was not affected by FasL and DetaNONOate. Red=CD31, blue=DAPI nuclei. Scale bar: 100 μm.

FIG. 7 depicts data from EVAc polymer slabs released NO donor (DetaNONOate) and FasL for two weeks.

FIGS. 8A-8D, depicts exemplary pig coronary arteries were medially injured and cultured statically. FIG. 8, panel A depicts an exemplary freshly isolated artery. FIG. 8B depicts an exemplary artery that was cultured for 7 days with none-releasing polymer slabs. FIG. 8C depicts an exemplary artery cultured with FasL- and DETANO-releasing polymer slabs at day 7. FIG. 8D depicts data demonstrating lumen area:vessel area ratios for three conditions: FasL- and DETANO-releasing slabs prevented neointimal thickening.

FIG. 9, comprising panels A through I, depicts results demonstrating that suspending FasL- and DETANO-releasing slabs in the culture media resulted in dramatic SMC death (Green, shown in panels A, D, and G depicts TUNEL) in the intima and media region while no apoptosis was observed in ECs (Yellow, shown in panels B, E, and H depicts von Willebrand Factor) after seven days.

FIG. 10 depicts exemplary balloon-expandable cobalt chromium alloy stents that were coated with NO donor and FasL carrying EVAc polymer.

FIG. 11, comprising panels A through D, depicts exemplary pig coronary arteries that were cultured ex vivo in perfusion bioreactors with 25 ml/min with either a non-releasing control stent (panels A and C) or a FasL- and NO donor-releasing stent (panels B and D). Lumen areas were larger and the vessel walls were thinner in the second group. The regions pointed with the red arrows are histological artifact.

FIG. 12, comprising panels A through E, depicts results demonstrating that a FasL- and NO donor-releasing stent (FIG. 12, panels C and D) caused significantly higher apoptosis in SMCs of pig coronary arteries cultured ex vivo with 25 ml/min flow compared with control stent (panels A and B). Green staining depicts TUNEL staining (shown alone in FIG. 12, panels A and C). Blue staining depicts DAPI staining (shown in FIG. 12, panels B and D, overlayed with TUNEL staining). FIG. 12 panel E shows that a FasL- and NO Donor-releasing stent results in significantly higher percentage of TUNEL+ cells indicating SMC apoptosis in the stent proximity.

FIG. 13 is a series of images illustrating NO Donor- and FasL-encapsulating PLGA particles can be positively charged by a chitosan layer, and then coated onto metal stents using DC current.

### DETAILED DESCRIPTION OF THE INVENTION

The present invention provides compositions and devices that release and/or elute a nitric oxide (NO) donor and a molecule that interacts with the Fas pathway. In certain embodiments, the invention includes a stent that releases and/or elutes a NO donor or NO and Fas ligand (FasL). In other embodiments, the invention includes a drug delivery agent that releases a NO donor or NO and FasL.

In certain embodiments, the invention includes methods wherein the NO and FasL releasing stents and/or drug delivery agents are administered to a subject to treat a condition in the subject. The treatments are, in certain embodiments, effective for conditions requiring a localized and selective effects. The compositions and methods of the invention are also effective for treating conditions, such as intimal hyperplasia, that require killing, or preventing growth of, smooth muscle cells and/or macrophages without affecting the growth or viability of endothelial cells.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, specific materials and methods are described herein. In describing and claiming the present invention, the following terminology will be used.

It is also to be understood that the terminology used herein is for the purpose of describing particular embodiments only, and is not intended to be limiting.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, “DetaNONOate” is a nitric oxide (NO) donor. It is also known as 2,2′-(2-Hydroxy-2-nitrosohydrazinylidene)bis-ethanamine, Diethylenetriamine NONOate, or NOC-18. DetaNONOate spontaneously decomposes in a pH-dependent, first-order process to liberate 2 moles of NO per mole of parent compound.

As used herein, “drug delivery agent” is any medium which facilitates in the delivery of a pharmaceutical compound(s) to a subject. In one embodiment, the drug delivery agent facilitates the delivery of a nitric oxide donor or nitric oxide and FasL.

“Fas ligand”, “FasL”, or “CD95L”, used interchangeably herein, is a type-II transmembrane protein and a member of the tumor necrosis superfamily. Upon binding its receptor, Fas (FasR, CD95), it forms a death-inducing signaling complex leading to apoptosis.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the term “stent” refers to a mesh tube that is inserted into the lumen of an anatomic vessel or duct to keep the passageway open. A stent can be one selected from the group of, but not limited to, a coronary stent, a vascular stent, a biliary stent, a ureteral stent, an arterial stent, or a venous stent. A stent may be made of a number of materials including plastics and metals and polyesters, and may be bio-degradable or non-degradable. A stent may be comprised of, for example but not limited to, nickel-titanium allow (e.g. “Nitinol”); stainless steel, magnesium, zinc, silicone rubber, nylon, polyesters including Dacron, polylactic acid, polyglycolic acid, polylactic-co-glycolic acid, polyetheroxide (PEO), polydimethylsiloxane (PDMS), polyhydroxylbuturate, or other materials that are suitable for deployment within the body that can provide some mechanical stenting function to maintain patency of tubular structures within the body.

As used herein, the term “subject,” “patient” or “individual” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, the terms “therapeutically effective amount”, “effective amount”, and “pharmaceutically effective amount” refer to a nontoxic but sufficient amount of an agent or drug to provide the desired biological result. That result can be reduction and/or alleviation of the signs, symptoms, or causes of a condition, disease, or disorder, or any other desired alteration of a biological system. An appropriate therapeutically effective amount in any individual case may be determined by one of ordinary skill in the art using routine experimentation.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### Stents

Metallic stents are often used to restore blood flow in stenotic vessels such as coronary arteries. However, stent deployment under high pressure leads to vascular wall and smooth muscle damage, including damage to cells within the media of the artery, with subsequent thrombosis, inflammation, and neointimal proliferation of SMCs. These events often result in vessel re-occlusion and treatment failure. In an effort to prevent intimal hyperplasia (i.e., thickening of blood vessels as a result of complications of a surgical or reconstruction procedure), drug-eluting stents (DES) were developed. These stents release global proliferation inhibitors, which hamper or stop the growth of SMCs as well as vascular endothelial cells (ECs), and typically mandate lifelong treatment with potent anti-coagulants. This is because proliferation inhibitors prevent re-endothelialization of the stent surface, leaving the clot-inducing metal exposed to the bloodstream for many years. Nitric oxide (NO)-releasing stents could in principle be safer than DESs, because in principle they could inhibit the growth only of SMC, but not of ECs. In certain non-limiting embodiments, NO acts primarily locally due to its very short diffusion distance. However, delivery of sufficient doses of NO to prevent intimal hyperplasia from a single stent has not been achieved to date in a stent in common clinical use.

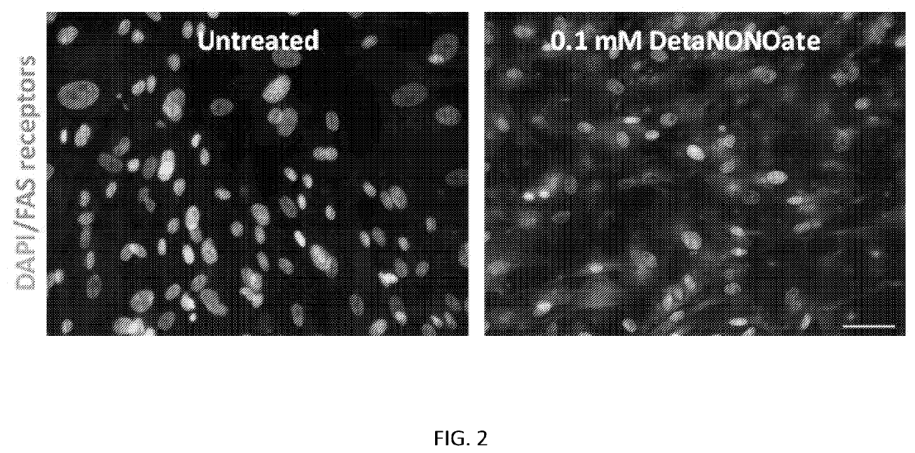

The present invention describes, for the first time, novel compositions and devices that increase the potency of NO and, when combined with another agent, inhibit SMC growth while retaining the survival and growth of the endothelium. The desired decrease in SMC growth and cell number is achieved at lower NO doses than would be possible from conventional, NO-releasing stents. As demonstrated herein, NO increases the expression of Fas receptors on the surface of SMCs preferentially over ECs (FIG. 2). Since NO has a short diffusion range (<200 μm), this effect of up-regulation of Fas-receptor is restricted to cells in close proximity to the stent which elaborates the NO. In certain embodiments, endothelial cells are resistant to Fas-mediated apoptosis, and are relatively resistant to increases in Fas-receptor due to local concentrations of NO. Therefore, NO can be used to enhance Fas-mediated SMC apoptosis, both locally and selectively, near the site of a deployed stent.

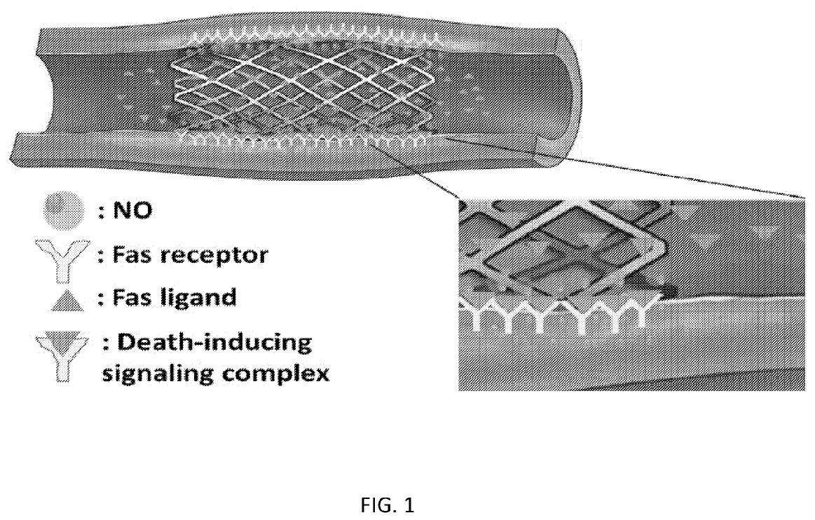

In certain embodiments, NO released and/or produced by the stent interacts only with SMCs that are in proximity of the stent struts, and increases Fas receptors on the surface of these cells. Fas ligand released by the stent binds to the Fas receptor, and forms a death-inducing signaling complex in SMCs that are close to vessel lumen. This effect differentially spares endothelial cells from Fas-mediated cell death (FIG. 1). Since SMCs are significantly more sensitive to Fas-mediated apoptosis than are endothelial cells (Boyle, et al., 2002, Arterioscler Thromb Vasc Biol 22(10):1624-30), delivery of both FasL and NO, as described herein, can stop intimal hyperplasia and stent failure, while retaining EC viability, much more efficiently than NO-releasing/eluting stents alone (FIG. 1). This mechanism can be used, for the first time, to selectively target SMCs and to prevent intimal hyperplasia without damaging re-endothelialization.

In one aspect, the invention includes a stent that contains or releases a nitric oxide (NO) donor and a molecule/agent that acts on the Fas pathway, for example molecules that interact with Fas receptors and which trigger apoptosis. Molecules acting on the Fas pathway include, but are not limited to, those which interact with and/or aggregate and/or trimerize the Fas receptor (FasR), such as Fas ligand (FasL), anti-FasR antibodies, siRNA, camptothecin, cisplatin, curcumin, ET-18-OCH3, resveratrol, TGF-β1, etoposide, vanadate, and vinblastine. In another aspect, the invention includes a composition comprising a stent wherein the stent releases a nitric oxide (NO) donor or nitric oxide and a Fas ligand (FasL). In certain embodiments, the FasL is a soluble, human form of FasL.

In certain embodiments of the stent, the NO donor comprises DetaNONOate. It should be noted that the NO donor contemplated in the present invention should be construed to include any molecule that provides a source of NO. Examples of NO donors include but are not limited to DetaNONOate, or sodium nitroprusside, as well as any other diazeniumdiolate or s-nitrosol type nitric oxide donor or nitric oxide conjugated proteins such as diazeniumdiolated- or s-nitrosylated-serum albumins or any bioactive material (e.g. 3,3-diselenodipropionic acid (SeDPA)) that produces nitric oxide by using endogenous donors present in the blood.

In certain embodiments, the action of the stent kills smooth muscle cells (SMCs) but not endothelial cells (ECs). In other embodiments, the stent acts locally. In yet other embodiments, the stent is a coronary artery stent.

In certain embodiments, the stents of the invention can be fabricated from one or more materials selected from the group consisting of metals, polymers, and plastics. The stents can include one or more metals and metal alloys selected from the group consisting of shape-memory alloys (e.g., nickel titanium (nitinol)), stainless steel, 316L stainless steel, cobalt-chromium alloy, nickel-cobalt-chromium alloy, tungsten, magnesium, platinum, iridium and tantalum. Other exemplary shape-memory alloys are described in publications such as Leonardo Lecce & Antonio Concilio, Shape Memory Alloy Engineering: For Aerospace, Structural and Biomedical Applications (2014). The stents can also include various non-metallic materials such as plastics such as polyethylene, polyurethane, polytetrafluoroethylene (PTFE), silicone, poly(propylene) (PP), polyethylene terephthalate (PET). The stents can also include one or more shape-memory polymers. Exemplary shape-memory polymers are described in publications such as Jinlian Hu, Shape Memory Polymers and Textiles (2007); Jinlian Hu, Shape Memory Polymers: Fundamentals, Advances and Applications (2014); and Jinsong Leng & Shanyi Du, Shape-Memory Polymers and Multifunctional Composites (2010). The stents are constructed using technologies known to one of skill in the art.

In certain embodiments, the stent is at least partially coated with ethylene-vinyl acetate copolymer (EVAc) (or any other biocompatible coating) that has been loaded with the nitric oxide (NO) donor and the Fas ligand (FasL). In other embodiments, the stent is coated with poly(lactic-co-glycolic acid) (PLGA) micro or nanoparticles (or any other bioabsorbable carriers) that encapsulate NO donor and the Fas ligand (FasL). In other embodiments, Fas ligand (FasL)-loaded polymer can be coated or patterned onto a stent surface made of bioactive material that produces NO by using the endogenous donors in the blood. In other embodiments, the NO donor and Fas ligand are loaded into degradable or non-degradable proteins such as, for example, collagen or fibrin, such that NO and Fas ligand are released from the protein coating over time. In other embodiments, the NO-donor and Fas ligand are incorporated into nanoparticles that are adhered to the stent surface. In other embodiments, molecules which release NO and Fas ligand are covalently tethered to the surface of the stent, which would then subsequently release the NO and Fas ligand over time.

### Medical Balloons

In certain embodiments of the invention, NO and Fas ligand are released from the surface of a balloon that is used for balloon angioplasty. Such balloons are typically used to dilate arteries or veins or airways or esophagus or other tubular structures within the body. Similar to stents, the media of the artery is typically injured during balloon dilatation, leading to intimal hyperplasia via similar mechanisms as are induced after stent deployment. Therefore, releasing NO and Fas ligand from the surface of a balloon that is used for arterial dilatation would have similar effects to those agents released from an arterial stent.

One aspect of the invention includes a medical balloon capable of releasing a nitric oxide (NO) donor, and/or NO, and an agent that aggregates and/or trimerizes a Fas receptor. In one embodiment, the agent is at least one selected from the group consisting of a Fas ligand (FasL), anti-FasR antibody, anti-FasR siRNA, camptothecin, cisplatin, curcumin, ET-18-OCH3, resveratrol, TGF-β1, etoposide, vanadate, and vinblastine. In one embodiment, the FasL is a soluble, human form of FasL. In another embodiment, the NO donor comprises DetaNONOate.

In certain embodiments, once the medical balloon is administered to a subject, release of the NO donor, and/or NO, and the agent kills smooth muscle cells (SMCs) and/or macrophages, but not endothelial cells (ECs) that are proximal to the administered stent.

In certain embodiments, the medical balloon is coated with a substance that releases the NO donor and/or NO, and the agent. Examples of such substances include but are not limited to a hydrophilic spacer (matrix carrier), a Poly(lactic-co-glycolic acid) (PLGA) nanoparticle, a Pluronic gel, and a protein. Types of proteins that can coat the medical balloon include but are not limited to collagen and fibrin. In one embodiment, the substance is covalently tethered to the medical balloon surface. Examples of hydrophilic spacers that can be used as medical balloon coatings include, but are not limited to, Iopromide and Urea. In another embodiment, the NO donor and/or NO, and the agent are released over time. In yet another embodiment, the NO donor and/or NO are released immediately.

### Drug Delivery Agents

In certain embodiments, the invention includes a composition or device comprising a drug delivery agent comprising a NO donor and a molecule that activates on the Fas pathway. Molecules acting on the Fas pathway include, but are not limited to, those that interact with or aggregate or trimerize the Fas receptor (FasR), such as Fas ligand (FasL), anti-FasR antibodies, siRNA, camptothecin, cisplatin, curcumin, ET-18-OCH3, resveratrol, TGF-β1, etoposide, vanadate, vinblastine. In certain embodiments, the invention includes a composition or device comprising a drug delivery agent comprising a NO donor and a FasL. In other embodiments, the FasL is a soluble, human form of FasL.

The NO donor of the present invention should be construed to include any molecule that provides a source of NO. Examples of NO donors include, but are not limited to, DetaNONOate, sodium nitroprusside, as well as any other diazeniumdiolate or s-nitrosol type nitric oxide donor or nitric oxide conjugated proteins such as diazeniumdiolated- or s-nitrosylated-serum albumins or any bioactive material (e.g. 3,3-diselenodipropionic acid (SeDPA)) that produces nitric oxide by using endogenous donors present in the blood. In certain embodiments, the NO donor comprises DetaNONOate.

In certain embodiments of the invention, the drug delivery agent kills smooth muscle cells (SMCs), macrophages, and/or other infiltrating cells that are sensitive to Fas-mediated apoptosis but not endothelial cells (ECs), or kills ECs to a substantially lesser degree than it kills SMCs. In other embodiments, the drug delivery agent acts locally.

In other embodiments, NO and Fas ligand might be released from a local polymer or gel or coating, whether biologically-derived or synthetic, that is applied to the inside of a blood vessel or other tubular structure within the body. Local release of NO and Fas ligand from a luminal surface coating of polymer or gel or sheet-like material in the inner lumen of an artery would be expected to have the same effects as those described for an arterial stent.

In other embodiments, NO and Fas ligand might be injected locally into an artery, vein, airway, or other tubular tissue in the body, with the intent of suppressing growth of smooth muscle cells while sparing other cell types. This type of embodiment might involve, for example, the injection of a combination of NO or NO-releasing drugs with Fas ligand or some Fas ligand analogue, into a coronary artery that has previously been treated with a stent or with balloon angioplasty. In such embodiments, the injected combination of drugs would be delivered into the lumen of the artery, vein, airway, etc., so as to act locally on SMCs near the lumen of the tubular structure in the body, thereby inhibiting growth and/or survival of SMCs that are near the lumen in those tubular structures.

### Methods of Treatment

Also included in the invention are methods for treating a condition in a subject in need thereof. In one aspect, the subject is administered a stent, wherein the stent releases a nitric oxide (NO) donor and a Fas ligand (FasL). In another aspect, the subject is administered a therapeutically effective amount of a drug delivery agent comprising a nitric oxide (NO) donor and a Fas ligand (FasL).

In certain embodiments, the NO comprises DetaNONOate. In other embodiments, the subject is administered a soluble, human form of FasL. However, the methods of the present invention should be construed to include treatment with any molecule or substance that interacts or interferes with the Fas pathway, in combination with a NO donor. Such molecules include, but are not limited to, FasL, antibodies that bind to FasR, and agents that trimerize the FasR.

The methods of the present invention should be construed to treat any condition wherein smooth muscle cells (SMCs) and/or macrophages, but not endothelial cells (ECs), should be killed (or their proliferation hampered or stymied). In certain embodiments, the condition is intimal hyperplasia. Other conditions that can be treated by the methods of the present invention include but are not limited to restenosis, anastomosis, gastric outlet syndrome, coronary artery disease, atherosclerosis, intimal hyperplasia, neointimal hyperplasia, pseudointimal hyperplasia, inflammation, transplantation-induced immunity, diabetes, hypertension, and conditions wherein the SMCs are exceedingly hypertrophic and/or hyperproliferative

The stents or drug delivery agents that release the present invention can be administered to any part of the subject that requires treatment. In certain embodiments, the stent is a coronary artery stent, and is administered to the arteries on the surface of the heart of the subject. In other embodiments, the invention is deployed within an artery, a vein, a urinary conduit such as ureter or urethra, an airway such a trachea or a mainstem bronchus or a bronchial airway, or a gastrointenstinal structure such as the pylorus, the duodenum, the esophagus, or the gastro-esophageal junction. In certain embodiments, the stent or drug delivery agent acts locally. In certain embodiments of the methods, the subject does not require treatment with an anti-coagulant following treatment with the invention.

The stents of the present invention can be constructed by methods commonly known to one of ordinary skill in the art. In certain embodiments, the stent is coated with ethylene-vinyl acetate copolymer (EVAc) that has been loaded with a nitric oxide (NO) donor and a Fas ligand (FasL). In other embodiments, the stent is coated with Poly(lactic-co-glycolic acid) (PLGA) nanoparticles that encapsulate a nitric oxide (NO) donor and a Fas ligand (FasL).

### Kits

The invention further provides kits comprising the elements disclosed elsewhere herein. A set of instructional materials can also be provided in the kit. The instructional materials can contain written, pictorial, and/or video directions on using the materials of the kit, including the methods of the invention.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### The Combination of FasL and NO Induces Apoptosis in Smooth Muscle Cells (SMCs) without Damaging Endothelial Cells (ECs)

Both local delivery of FasL, and increasing the Fas receptors on the surface of the target cells, can increase the efficiency of FasL-mediated apoptosis. Nitric oxide (NO) is known to increase surface Fas receptors on vascular SMCs (Fukuo et al., Hypertension, 1996. 27(3 Pt 2): p. 823-6; Boyle, Weissberg, and Bennett, Arterioscler Thromb Vasc Biol, 2002. 22(10): p. 1624-30). This was demonstrated herein when cultured human aortic SMCs were treated with the NO donor DetaNONOate. After 24 hours of treatment with DetaNONOate, Fas receptor expression on SMCs was increased (red stain, FIG. 2, right panel).

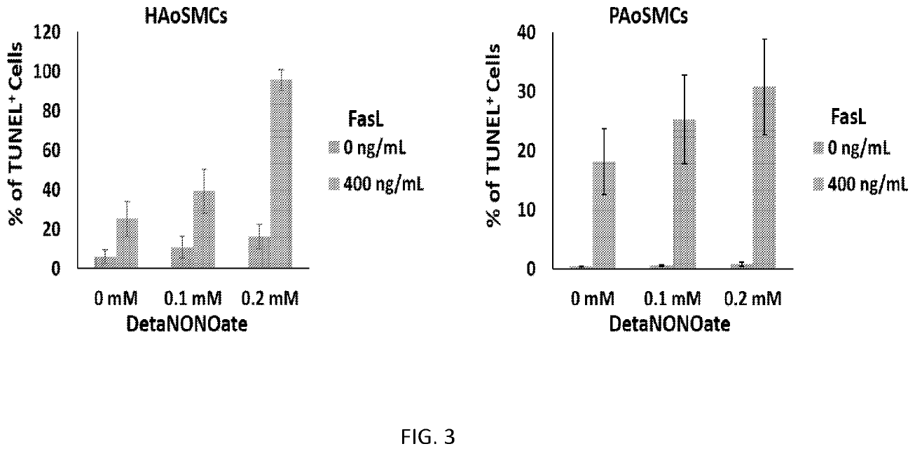

Subsequently, cell culture studies demonstrated that local delivery of FasL and NO synergistically induced apoptosis in SMCs (FIG. 3). NO has a very short diffusion length of <200 μm in tissues—therefore, NO can induce an upregulation of Fas receptor on SMC only within a target area that is very close to the stent surface. This, in turn, limits the potential for broader tissue damage, since the SMC Fas receptors are increased only locally. Therefore, co-release of NO and FasL near the SMCs in the arterial wall affects only those SMCs in close proximity to the released agents.

Addition of FasL and NO donor DetaNONOate increased human and pig aortic SMC apoptosis in a synergistic fashion (FIG. 3). DetaNONOate alone, up to 0.2 mM concentration, led to minimal (<15%) death of human or porcine SMCs. In contrast, the addition of FasL at 400 ng/mL greatly enhanced SMC killing of NO. TUNEL staining showed 30% killing of porcine SMCs, and nearly 100% killing of human SMCs, after 48 hours at these particular doses. These data demonstrated the synergistic effects of FasL+NO, and show that the combination of agents is more effective at inducing SMC apoptosis than NO alone.

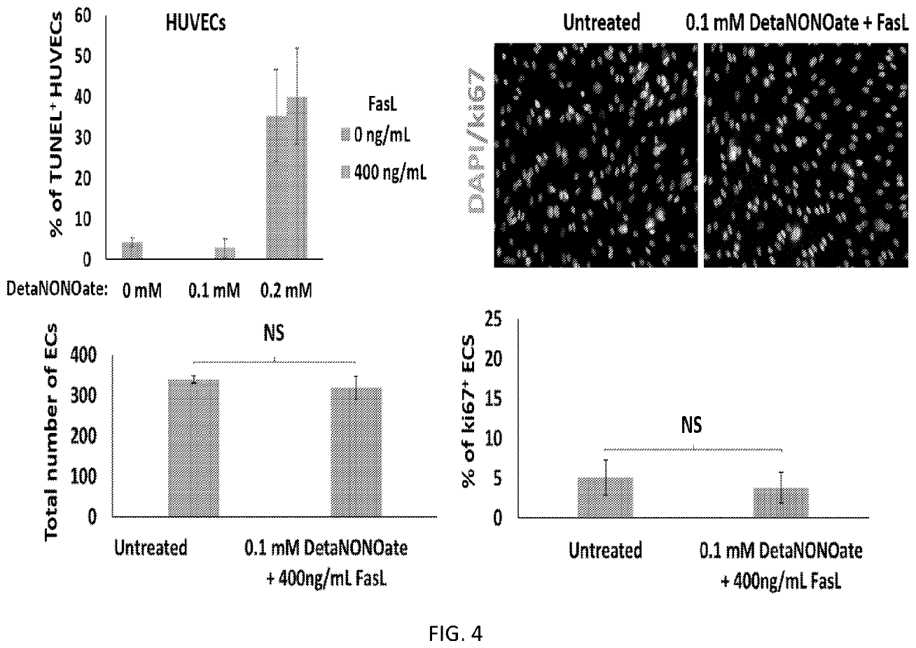

Next, the effect of FasL and NO donor combinations on EC viability was tested. Addition of DetaNONOate alone at 0.1 mM concentration did not affect human umbilical vein endothelial cell (HUVEC) cell number, rates of apoptosis or proliferation, in the absence of FasL (FIG. 4). When FasL at 400 ng/mL was added to HUVEC cultures that were exposed to 0.1 mM concentration DetaNONOate, again no significant change in cell number, apoptosis or proliferation was observed (FIG. 4). These data, combined with those of FIG. 3, showed that a dosage combination of 0.1 mM DetaNONOate and 400 ng/mL FasL had a selective and differential effect on the survival of cultured porcine and human SMCs and ECs. Of note, higher concentrations of DetaNONOate at 0.2 mM negatively affected EC viability, showing the importance of tuning the dosage range for these two drugs when optimizing stent function.

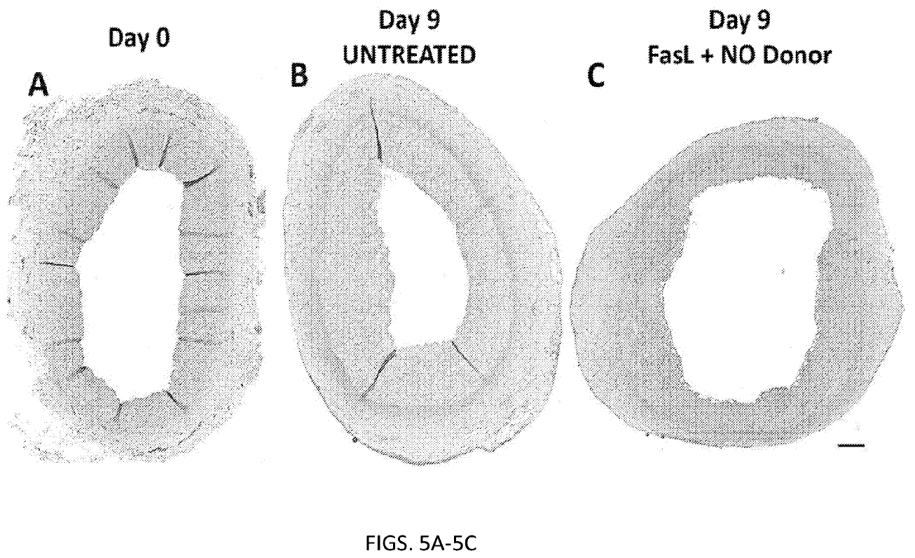

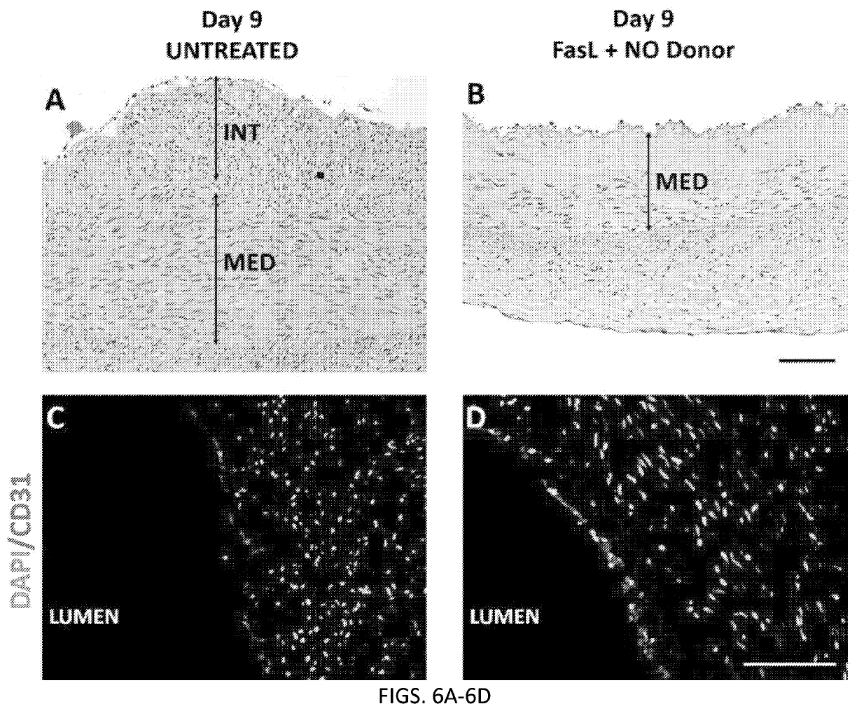

To further delineate the potential effects of the co-delivery of NO and FasL in native arteries, an ex vivo coronary artery culture model was created. Freshly harvested porcine coronary arteries were injured by inserting a 2 mm-thick needle into the lumen, which distended the wall of the vessel acutely and which damaged SMC within the media of the artery to mimic vessel wall injury and to trigger SMC proliferation. After removal of the needle, the coronary arteries were cultured under standard culture conditions in Dulbeccos Modified Eagles Medium (DMEM) with 10% fetal bovine serum (FBS), (DMEM/FBS), and histological outcomes after nine days were assessed (FIGS. 5A-5C). Addition of FasL and DetaNONOate to the culture medium was compared to control culture conditions. In injured coronary arteries that were cultured for nine days in DMEM/FBS, the intima region became noticeably thicker as compared to freshly excised coronary arteries (FIGS. 5A-5B, FIG. 6A). However, addition of 400 ng/mL FasL and 0.1 mM DetaNONOate to the culture medium, beginning on day five and continuing through day nine, largely prevented intimal thickening (FIG. 5C, FIG. 6B). The FasL-NO donor combination decreased SMCs in the medial region, and created occasional acellular regions within the media of the artery (FIG. 6B). However, CD31 staining showed that ECs remained on the lumen, implying the lack of toxicity to endothelial cells (FIGS. 6C-6D).

In summary, data described herein showed at least the following: NO increases Fas receptors on SMCs and enhances FasL-mediated SMC apoptosis, NO concentrations can be optimized to spare ECs from Fas-mediated apoptosis, and releasing NO and FasL from a stent surface should inhibit intimal hyperplasia, in-stent stenosis and late thrombosis, while sparing ECs.

### Example 2

### Drug Delivery and Stent Designs

One embodiment of the invention includes drug delivery using a stent with a biocompatible/nonabsorbable polymer coating. Ethylene-vinyl acetate copolymer (EVAc) is an FDA-approved polymer for drug delivery applications. It can be loaded with proteins such as nerve growth factor and albumin, and can provide sustained release of these proteins (Powell et al., Brain research 1990; 515(1-2): 309-11). In one embodiment of the present invention, EVAc is loaded with a nitric oxide donor (or nitric oxide conjugated proteins such as diazeniumdiolated- or s-nitrosylated-serum albumins) and Fas ligand (or any other molecule which interacts with Fas receptors and triggers apoptosis). The stents are then coated with the drug-loaded EVAc, such that the drugs (NO and Fas ligand) can be delivered over time from the surface of the stent.

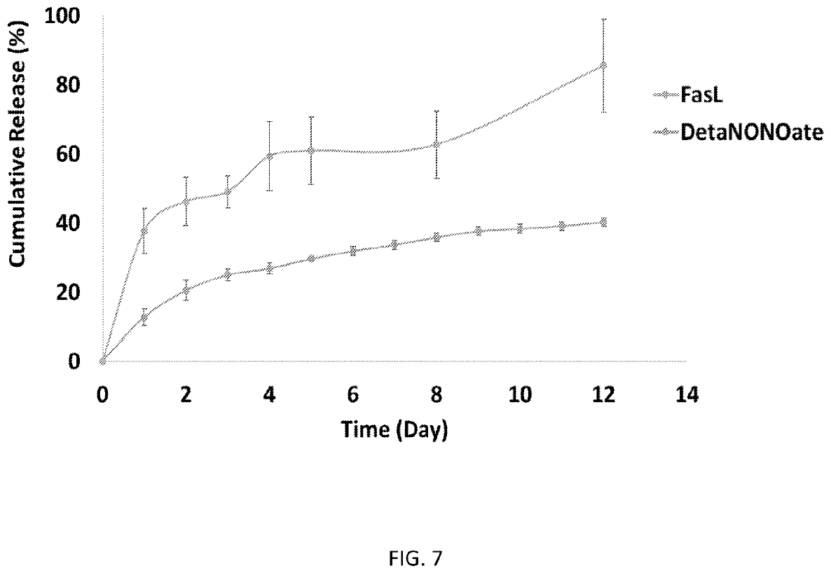

Herein, NO donor DetaNONOate and recombinant human Fas ligand were mixed with Ficoll to obtain an inert powder, and EVAc polymer was doped with this drug mixture. When incubated in PBS at 37° C., EVAc polymer slabs released both FasL and DetaNONOate persistently over two weeks, without the undesirable “burst effect” that is sometimes observed with polymeric drug encapsulation (FIG. 7).

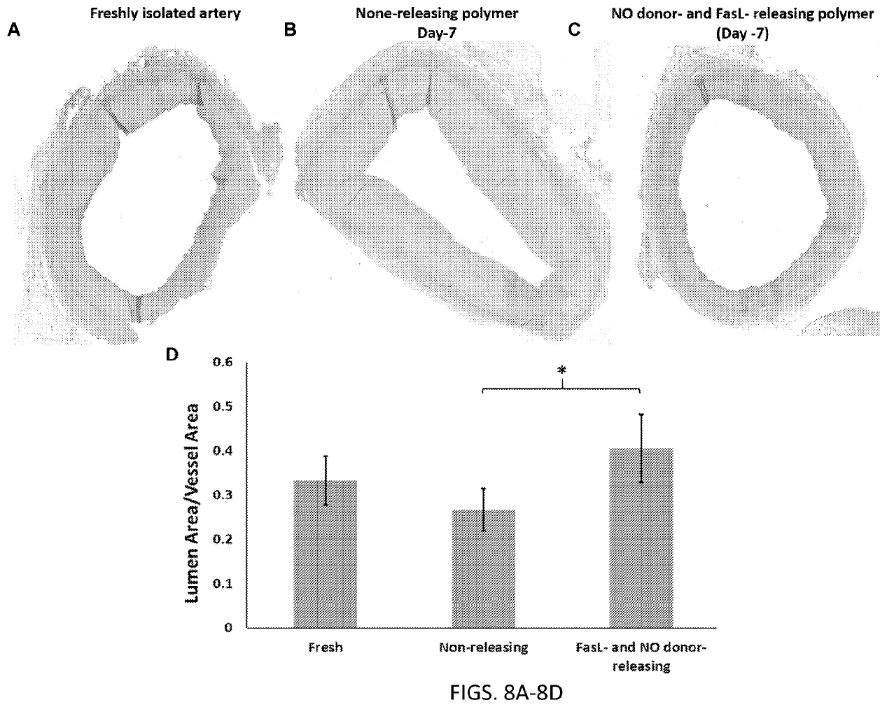

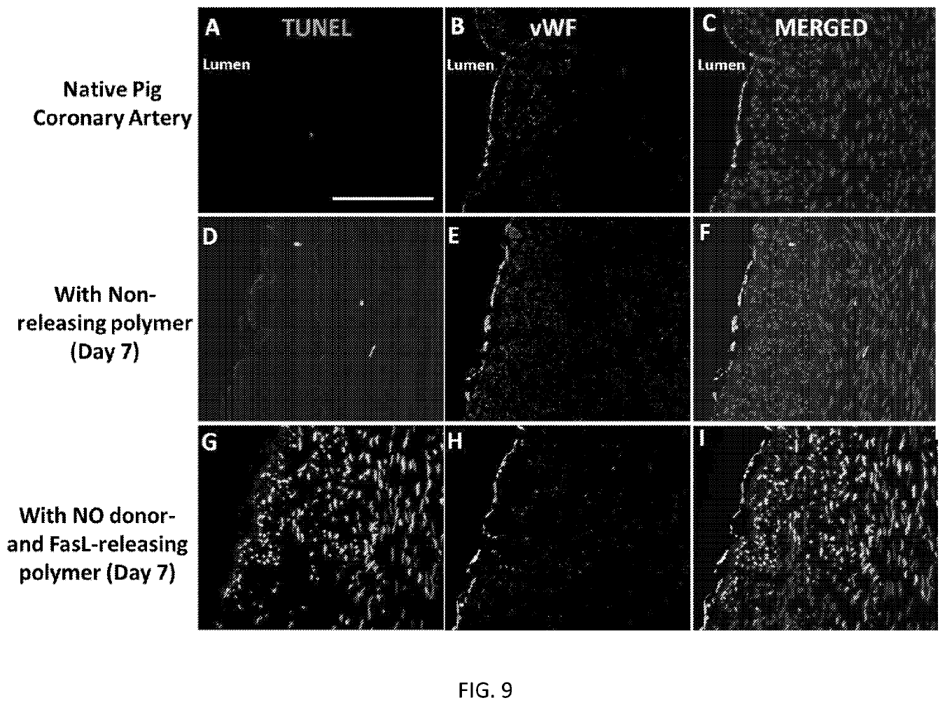

Next, freshly harvested porcine coronary arteries were cultured under standard culture conditions in DMEM:Vasculife (50:50) with 20% FBS with EVAc polymer slabs suspending in the culture media, histological outcomes were assessed after 7 days (FIGS. 8A-8D). Suspension of FasL- and DETANO-releasing EVAc slabs in the culture medium was compared to control culture conditions. In injured coronary arteries that were cultured for 7 days with only-Ficoll-releasing control slabs, the intima region became noticeably thicker as compared to freshly excised coronary arteries (FIGS. 8B and 8D). However, FasL- and Deta NO-releasing EVAc slabs largely prevented intimal thickening (FIGS. 8C and 8D). TUNEL staining showed that FasL-NO donor released from the slabs increased SMC apoptosis in the intima and media region (green stain, FIG. 9, panels A, D and G). However, vWF (von Willebrand factor) staining and absence of the TUNEL showed that EC remained on the lumen, implying lack of toxicity to this cell type (yellow stain, FIG. 9, panels B, E and H).

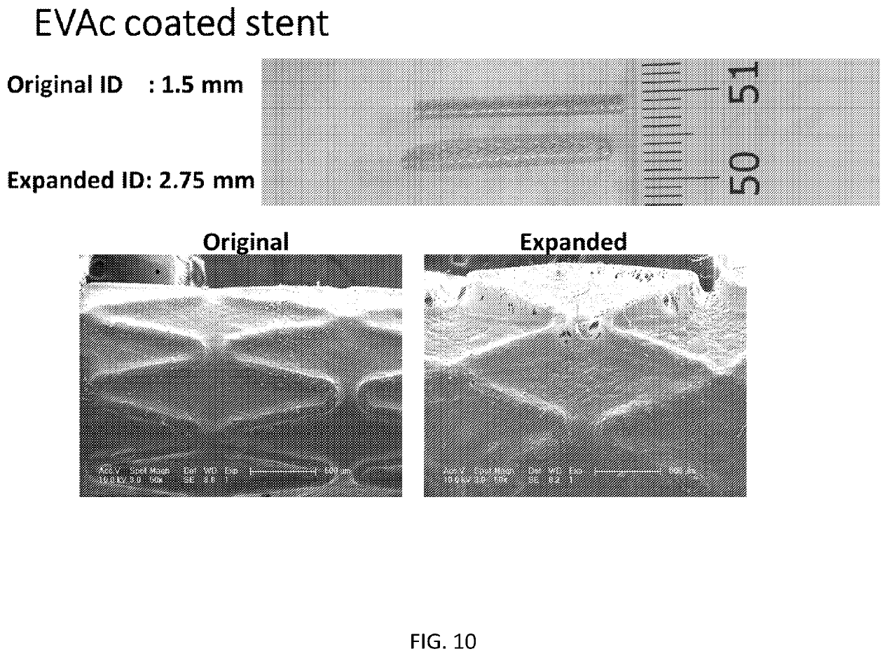

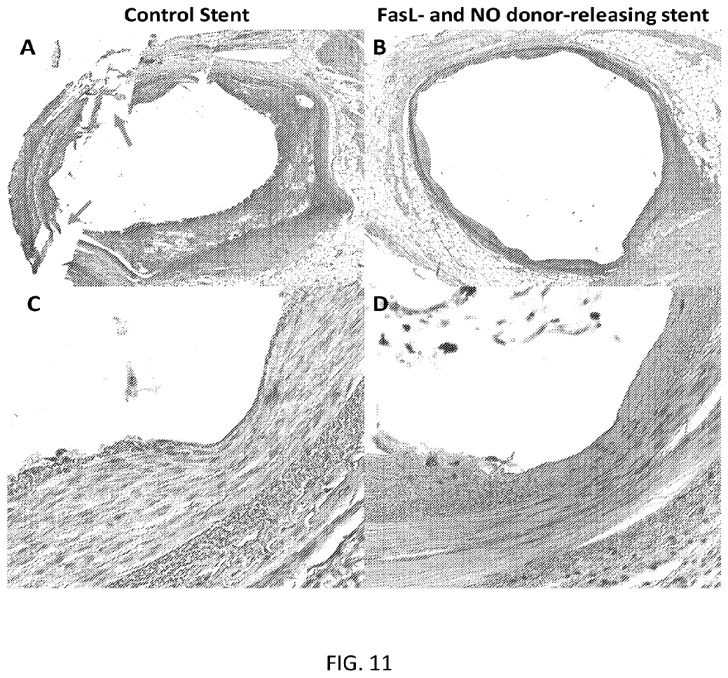

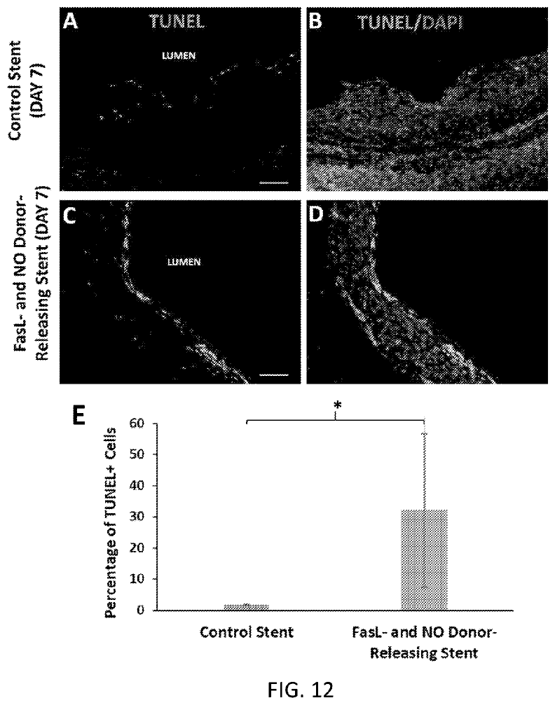

Finally, herein, cobalt chromium (CoCr) alloy stents were coated with EVAc loaded with DetaNONOate and FasL (FIG. 10), and were deployed in the lumens of freshly isolated pig coronary arteries. Stents with no drugs were used as control. The stented pig coronary arteries were cultured in perfusion bioreactors with 25 mL/min flow rate (mimicking porcine coronary flow rate) for one week (culture medium was perfused through the vessel lumen by the help of peristaltic pumps). The mass of EVAc coating on each stent was approximately 1.6 mg, and was easily loaded onto the stent struts. At the end of one week, arteries with FasL- and NO donor-releasing stents had larger lumen area-to-vessel area ratios (0.58±0.13 versus 0.73±0.13) and there were acellular regions in the media region near the stent struts (FIG. 11). These results show that stents can locally deliver functional amounts of FasL and NO donor to the vessel wall even when there is physiological flow of fluid through the coronary vessel lumen. TUNEL stain (green) in FIG. 12 shows that co-release of FasL and NO donor resulted significantly higher SMC apoptosis in the stent proximity, indicating the local effect of the released drugs.

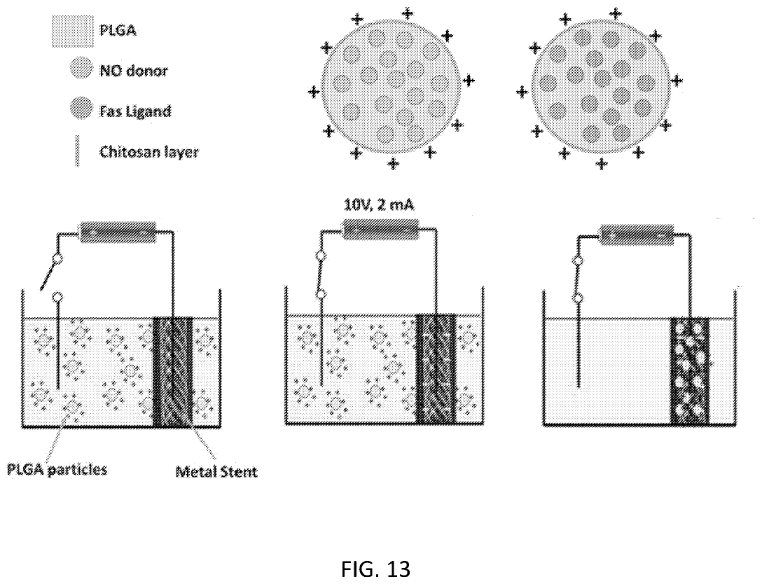

Another embodiment includes drug delivery with biodegradable polymer particles. Poly(lactic-co-glycolic acid) (PLGA) is an FDA-approved bioabsorbable polymer. PLGA nanoparticles (NPs) provide efficient delivery of different classes of therapeutic agents such as proteins, oligonucleotides, DNAs or siRNAs (Panyam et al., FASEB journal: official publication of the Federation of American Societies for Experimental Biology 2002; 16(10): 1217-26; Zhou et al., Biomaterials 2012; 33(2): 583-91; Fahmy et al., Biomaterials 2005; 26(28): 5727-36). Persistent therapeutic effects can be obtained by controlling the release of agents that are encapsulated by such nanoparticles. NO donor-encapsulating PLGA particles can sustain release of NO in concentrations comparable to healthy endothelium over 4 weeks following an initial burst release (Lautner et al., Journal of controlled release: official journal of the Controlled Release Society 2016; 225: 133-9; Do et al., Radiology 2004; 230(2): 377-82; Acharya et al., Journal of biomedical materials research Part A 2012; 100(5): 1151-9). In certain embodiments of the present invention, metal stents are coated with Fas ligand- and NO donor-encapsulated PLGA NPs using cationic electrodeposition by creating positive charges on the particle surface (Nakano, et al., JACC Cardiovascular interventions 2009; 2(4): 277-83; Tsukie et al., Journal of atherosclerosis and thrombosis 2013; 20(1): 32-45) (FIG. 13).

In certain embodiments, the Fas ligand and NO donor are encapsulated in the PLGA particles by a water-oil-water double emulsion solvent evaporation method (Zhou et al., Biomaterials 2012; 33(2): 583-91; Fahmy et al., Biomaterials 2005; 26(28): 5727-36; Acharya et al., Journal of biomedical materials research Part A 2012; 100(5): 1151-9). Particle size, homogeneity of the polymer-drug mixture, polymer and drug concentration, lactide to glycolide (L/G ratio) in PLGA, and addition of surfactants can affect the release profile of the drug from NPs (Taghipour et al., Research in pharmaceutical sciences 2014; 9(6): 407-20; Giteau et al., International journal of pharmaceutics 2008; 350(1-2): 14-26; Han et al., Frontiers in Pharmacology 2016; 7(185)). Maintaining the local concentrations of the NO donor and FasL concentrations (for example, ≈0.1 mM and 400 ng/mL, respectively) until the repopulation of endothelium (4 weeks) is helpful in preventing neointimal hyperplasia formation, by initiating apoptosis in SMCs and infiltrating immune cells that accumulate on the stented lumen. The stents of the present invention can be coated with any particles encapsulating NO donor (or any NO-releasing compound) and Fas ligand (or any other molecule which interacts with Fas receptors and triggers apoptosis).

### Example 3

Herein, a novel drug-eluting stent was designed that prevents intimal hyperplasia by inducing apoptosis in SMCs that are in proximity to the stent struts only, without preventing EC proliferation. The combined release of NO and FasL from the stent surface kills the cells migrating to the stented vessel lumen, locally and selectively, and prevents intimal hyperplasia without affecting re-endothelialization of the stent surface. The significance of this work is a novel drug-eluting stent which, unlike bare metal stents and commercially available drug-eluting stents (DES), has fewer short-term and long-term complications such as in-stent restenosis and late thrombosis. Stenting is one of the most frequently used percutaneous coronary interventions to restore the circulation in stenotic coronary vessels. However, restenosis rates with bare metal stents were reported to be between 16% and 44%, depending on the lesion length and vessel caliber. Hence, by 2007, 95% of the stents used in the US and Europe were DES, which now typically mandate lifelong treatment with potent anti-coagulants (Newsome et al., Anesthesia & Analgesia, 2008. 107(2): p. 552-569). As demonstrated herein, NO can enhance Fas-mediated apoptosis of SMCs, thus preventing intimal hyperplasia. This approach exploits differences in FasL-mediated cell death between SMC and endothelium. Moreover, it utilizes the short diffusion range of NO as a very effective tool to limit the target region affected by the ligand, which is an added advantage over currently used DES.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A stent capable of releasing (a) a nitric oxide (NO) donor and/or NO, and (b) an agent that aggregates and/or trimerizes a Fas receptor,
wherein the agent comprises at least one selected from the group consisting of a Fas ligand (FasL), anti-FasR antibody, anti-FasR siRNA, camptothecin, cisplatin, curcumin, ET-18-OCH3, resveratrol, TGF-β1, etoposide, vanadate, and vinblastine,
wherein the stent is coated with a substance that releases (a) the NO donor and/or NO, and (b) the agent, wherein the substance is selected from the group consisting of an ethylene-vinyl acetate copolymer (EVAc), a Poly(lactic-co-glycolic acid) (PLGA) nanoparticle, a Pluronic gel, and a protein.

2. (canceled)

3. (canceled)

4. The stent of claim 1, wherein the FasL comprises a soluble, human form of FasL.

5. The stent of claim 1, wherein the NO donor comprises DetaNONOate.

6. (canceled)

7. (canceled)

8. (canceled)

9. The stent of claim 1, wherein the protein is selected from the group consisting of collagen and fibrin.

10. (canceled)

11. (canceled)

12. (canceled)

13. A medical balloon capable of releasing (a) a nitric oxide (NO) donor and/or NO, and (b) an agent that aggregates and/or trimerizes a Fas receptor,
wherein the agent comprises at least one selected from the group consisting of a Fas ligand (FasL), anti-FasR antibody, anti-FasR siRNA, camptothecin, cisplatin, curcumin, ET-18-OCH3, resveratrol, TGF-β1, etoposide, vanadate, and vinblastine.

14. (canceled)

15. (canceled)

16. The medical balloon of claim 13, wherein the FasL comprises a soluble, human form of FasL.

17. The medical balloon of claim 13, wherein the NO donor comprises DetaNONOate.

18. (canceled)

19. (canceled)

20. The medical balloon of claim 13, wherein the medical balloon is coated with a substance that releases (a) the NO donor and/or NO, and (b) the agent, wherein the substance is selected from the group consisting of an ethylene-vinyl acetate copolymer (EVAc), a Poly(lactic-co-glycolic acid) (PLGA) nanoparticle, a Pluronic gel, and a protein.

21. The medical balloon of claim 20, wherein the protein is selected from the group consisting of collagen and fibrin.

22. (canceled)

23. (canceled)

24. (canceled)

25. A method of treating a condition in a subject, the method comprising administering to the subject a composition capable of releasing within the subject (a) a NO donor and/or NO, and (b) an agent that aggregates and/or trimerizes a Fas receptor, wherein the composition is selected from the group consisting of a stent, a medical balloon, a hydrophilic spacer, a polymer, and a gel.

26. The method of claim 25, wherein the NO comprises DetaNONOate.

27. The method of claim 25, wherein the agent comprises a FasL, wherein the FasL comprises a soluble, human form of FasL.

28. (canceled)

29. (canceled)

30. (canceled)

31. The method of claim 25, wherein the composition is coated with a substance that releases (a) the NO donor and/or NO, and (b) the agent, wherein the substance is at least one selected from the group consisting of a hydrophilic spacer, a Pluronic gel, Poly(lactic-co-glycolic acid) (PLGA), and a protein.

32. The method of claim 31, wherein the protein is selected from the group consisting of collagen and fibrin.

33. (canceled)

34. (canceled)

35. (canceled)

36. The method of claim 25, wherein the polymer or gel is applied inside a blood vessel.

37. The method of claim 25, wherein the condition is selected from the group consisting of intimal hyperplasia, restenosis, anastomosis, gastric outlet syndrome, coronary artery disease, atherosclerosis, neointimal hyperplasia, pseudointimal hyperplasia, inflammation, transplantation-induced immunity, diabetes, hypertension, and conditions wherein the SMCs are hypertrophic and/or hyperproliferative.

38. The method of claim 25, wherein the subject is human.

39. (canceled)

40. A drug delivery composition comprising (a) a NO donor and/or NO, and (b) an agent that aggregates and/or trimerizes the Fas receptor,
wherein the NO donor comprises DetaNONOate,
wherein the agent comprises a FasL; and,
wherein the composition comprises a polymer or a gel.

41. (canceled)

42. (canceled)

43. The composition of claim 40, wherein the FasL comprises a soluble, human form of FasL.

44. (canceled)

45. (canceled)

46. (canceled)

47. A kit comprising the composition of claim 40 and instructional material for use thereof.
